(19) United States
(12) Patent Application Publication
(10) Pub. No.: US 2014/0228682 A1
(43) Pub. Date: Aug. 14, 2014

(54) IMAGING PROBES, IMAGING SYSTEMS, AND METHODS OF IMAGING

(71) Applicant: The Board of Trustees of the Leland Stanford Junior University, Palo Alto, CA (US)

(72) Inventors: Hongguang Liu, Liaoning (CN); Xiang Hu, Hunan (CN); Zhen Cheng, Mountain View, CA (US); Yang Liu, Hangzhou (CN)

(21) Appl. No.: 14/173,232

(22) Filed: Feb. 5, 2014

Related U.S. Application Data
(60) Provisional application No. 61/763,173, filed on Feb. 11, 2013.

(51) Int. Cl. A61B 6/00

(52) U.S. Cl. CPC A61B 6/5217; A61B 6/481; A61B 6/4057; A61B 6/425; A61B 6/44; A61B 2019/542
USPC 600/431; 600/436

(57) ABSTRACT

Embodiments of the present disclosure include methods of imaging a target area, methods of monitoring the degeneration of cartilage, and the like.

### CLAIM OF PRIORITY TO RELATED APPLICATION

This application claims priority to co-pending U.S. provisional application entitled “IMAGING PROBES, IMAGING SYSTEMS, AND METHODS OF IMAGING” having Ser. No.: 61/763,173, filed on Feb. 11, 2013, which is entirely incorporated herein by reference.

### BACKGROUND

There are various types of cartilage, e.g., hyaline cartilage and fibrocartilage. Hyaline cartilage, also referred to as articular cartilage, is found at the articular surfaces of bones, e.g., in the joints, and is responsible for providing the smooth, nearly frictionless, gliding motion characteristic of moveable joints. Articular cartilage is firmly attached to the underlying bones.

Adult cartilage has a limited ability of repair itself. As a result, damage to cartilage produced by disease or trauma can lead to serious physical deformity and debilitation. Articular cartilage is aneural, avascular, and alymphatic, so ariticular cartilage can be clinically and radiographically silent (MRI, PET, CECT and optical imaging). In addition, high resolution images of the boundary region between bone and cartilage can be difficult to obtain. Thus, there is a need to develop imaging techniques to evaluate cartilage.

### SUMMARY

Embodiments of the present disclosure include methods of imaging a target area, methods of monitoring the degeneration of cartilage, and the like.

In an embodiment, a method of imaging a target area, among others, can include: introducing a probe into a subject or a sample, where the probe is capable of emitting positrons and photons; detecting gamma rays generated by the positrons emitted from the probe; and detecting low energy photons generated by the probe, where the origin of the gamma ray and the photons corresponds to the location of the probe, where the location of the probe corresponds to the location of the target area.

In an embodiment, a method of monitoring the degeneration of cartilage, among others, can include: introducing a probe into a subject or a sample at a first time, wherein the probe is capable of emitting positrons and photons; detecting gamma rays generated by the positrons emitted from the probe; detecting low energy photons generated by the probe; generating a first image of a target area that includes the cartilage, wherein the origin of the gamma ray and the photons corresponds to the location of the probe, wherein the location of the probe corresponds to the location of the target area; repeating the steps above at a second time to generate a second image corresponding to the second time; and comparing the images produced at the first time and the second time to monitor the degeneration of the cartilage.

In an embodiment, a method of imaging articular cartilage, among others, can include: introducing ⁸⁹Zr oxalate into a subject, wherein the ⁸⁹Zr ion is capable of emitting positrons and photons; detecting gamma rays generated by the positrons emitted from the ⁸⁹Zr ion; and detecting low energy photons generated by the ⁸⁹Zr ion, wherein the origin of the gamma ray and the photons corresponds to the location of the ⁸⁹Zr ion, wherein the location of the ⁸⁹Zr ion corresponds to the location of the articular cartilage.

Other systems, methods, features, and advantages will be, or become, apparent to one with skill in the art upon examination of the following drawings and detailed description. It is intended that all such additional structures, systems, methods, features, and advantages be included within this description, be within the scope of the present disclosure, and be protected by the accompanying claims.

### BRIEF DESCRIPTION OF THE DRAWINGS

Many aspects of this disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an embodiment of a probe,⁸⁹Zr oxalate.

FIG. 2 illustrates that the ⁸⁹Zr oxalate can specifically image the osteoarthritis and differentiate it from shamed and untreated joints (high signal in sham and untreated joints, whereas low signal in OA joints).

FIG. 3 illustrates the same information that the ⁸⁹Zr oxalate can specifically image the osteoarthritis and differentiate it from shamed and untreated joints.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of imaging, chemistry, synthetic organic chemistry, biochemistry, biology, molecular biology, microbiology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted.

The term “molecular imaging”, as used herein, relates to the in-vivo characterization and measurement of biologic processes and pathways at the cellular and molecular levels.

The term “optical imaging”, as used herein, relates to the generation of images by using photons in a wavelength range (e.g., ultraviolet to infrared). The term “optical imaging”, “optical imaging”, and “Cerenkov imaging” as used herein, relate to the detection of optical signals generated by radiolabelled or radioactive probes (also referred to as “probe”).

The term “multiplexed detection”, as used herein, relates to the simultaneous detection and differentiation of multiple signals from the same probe.

The term “detectable” refers to the ability to detect a signal over the background signal.

The term “detectable signal” can refer to a signal derived (directly or indirectly (as in PET)) from a probe. The detectable signal is detectable and distinguishable from other background signals that are generated from the subject or sample. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between detectable signal and the background. Standards and/or calibration curves can be used to determine the relative intensity of the detectable signal and/or the background.

The term “signal” refers to a signal derived from a probe. The signal can be generated from one or more probes and can be in the form of an optical signal or gamma ray signal. In an embodiment, the signal may need to be the sum of each of the individual signals. In an embodiment, the signal can be generated from a summation, an integration, or other mathematical process, formula, or algorithm, where the signal is from one or more probes, or the like. In an embodiment, the summation, the integration, or other mathematical process, formula, or algorithm can be used to generate the signal so that the signal can be distinguished from background noise and the like. It should be noted that signals other than the signal of interest can be processed and/or obtained is a similar manner as that of the signal of interest.

The signal or energy can be detected and quantified in real time using an appropriate detection system such as those described herein.

The term “in vivo imaging” as used herein refers to methods or processes in which the structural, functional, or physiological state of a living being is examinable without the need for a life ending sacrifice.

The term “non-invasive in vivo imaging” as used herein refers to methods or processes in which the structural, functional, or physiological state of a being is examinable by remote physical probing without the need for breaching the physical integrity of the outer (skin) or inner (accessible orifices) surfaces of the body.

The term “sample” can refer to a tissue sample, cell sample, a fluid sample, and the like. The sample may be taken from a subject. The tissue sample can include brain, hair (including roots), buccal swabs, blood, saliva, semen, muscle, or from any internal organs, or cancer, precancerous, or tumor cells associated with any one of these. The fluid may be, but is not limited to, urine, blood, ascites, pleural fluid, spinal fluid, and the like. The body tissue can include, but is not limited to, brain, skin, muscle, endometrial, uterine, and cervical tissue or cancer, precancerous, or tumor cells associated with any one of these. In an embodiment, the body tissue is brain tissue or a brain tumor or cancer.

The term “administration” refers to introducing a probe of the present disclosure into a subject. One preferred route of administration of the compound is oral administration. Another preferred route is intravenous administration. However, any route of administration, such as topical, subcutaneous, peritoneal, intraarterial, inhalation, vaginal, rectal, nasal, introduction into the cerebrospinal fluid, or instillation into body compartments can be used.

As used herein, the term “subject,” or “patient,” includes humans and mammals (e.g., mice, rats, pigs, cats, dogs, and horses). Typical subjects to which probes of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive and not just a part excised (e.g., a liver or other organ) from the living subject.

### General Discussion

Embodiments of the present disclosure provide for probes, methods of using the probes, methods of detecting gamma rays and optical signals derived (directly or indirectly) from the probe, methods of imaging a disease or condition (e.g., cartilage degeneration), and the like. In an embodiment, the probe can function as a dual modality probe for PET imaging and Cerenkov imaging. Embodiments of the present disclosure can be used to image, detect, study, monitor, and/or evaluate, a condition or disease such as, but not limited to, cartilage degeneration (e.g., articular cartilage), in a subject or sample. In addition, embodiments of the present disclosure can be used for multiplexed imaging.

Embodiments of the present disclosure can be advantageous due to the high uptake of the probes, high sensitivity relative to other probes, high resolution imaging, and the like. In a particular embodiment, the probe accumulates in cartilage (e.g., articular cartilage), but has fast clearance from the body. In an embodiment, the accumulation supports the high ratio of the accumulation on healthy cartilage relative to degenerated cartilage, which enhances the images produced using probes of the present disclosure.

In particular, exemplary embodiments of the present disclosure encompass methods and systems for non-invasive in-vivo optical molecular imaging using a probe of the present disclosure that can provide high resolution images from a living subject or a sample.

Light is electromagnetic radiation, particularly radiation of a wavelength that is visible to the human eye (e.g., about 350-750 nm). Radionuclides, including alpha and beta emitters, are able to generate continuous spectra of photons by interaction with surrounding materials and therefore, can be monitored at different wavelengths. The lower energy photons associated with emitted charged particles during decay of radionuclides, corresponding to an energy below 0.005 keV and to wavelengths above about 300 nm, have been found to be highly suited for medical molecular imaging due to the achieved high sensitivity and spatial resolution.

Cerenkov luminescence imaging (CLI) has emerged as an active field of research in the biomedical community, since it offers the potential of cost-effective molecular imaging that combines the above-mentioned advantages of both nuclear medicine and optical imaging. Cerenkov light is originated when charged nuclear particles such as β⁺ (positron) or β⁻ (nuclear electron), emitted from radionuclides, travel at superluminal velocity in any dielectric medium such as biological tissue or water. Therefore CLI can be performed with positron emitters. Cerenkov radiation is continuous and occurs mainly in the visible (more intense in the blue) region of the electromagnetic spectrum in the wavelength range of about 400-1000 nm. This facilitates in vivo optical imaging of a subject such as a living subject intravenously administered with a probe, using commercially available optical imaging systems (e.g., IVIS 200 Spectrum, Caliper Life Sciences) that are equipped with cooled CCD cameras.

Compared to conventional fluorescence and bioluminescence imaging, radioactive optical imaging (OI) has some unique properties. The continued emission wavelength of radioactive OI allows monitoring and imaging of a radionuclide at different wavelengths, which is a significant advantage over the conventional optical imaging modalities. And the radioactive OI signal generated by a radionuclide does not require an excitation light and is always on, which is different from fluorescence and bioluminescence probes, which typically need an outside source of energy and which may produce unwanted and complicating optical signals from other sources (e.g., skin).

Gamma-rays-emission based imaging modalities, such as PET or SPECT imaging modalities, produce gamma rays through annihilation events by the positrons emitted by the PET or SPECT imaging modality. The gamma rays can be detected and correlated to the approximate location of the PET or SPECT imaging modality.

Since the origin and source of the signal are the same in both PET and CLI, the combined PET and Cerenkov detection can lead to a more accurate method of imaging using the same probe.

In an exemplary embodiment, a probe can function as a dual modality probe for PET imaging and a Cerenkov imaging. In an embodiment, the probe includes ⁸⁹Zr. ⁸⁹Zr has a half life of 78.4 hours and positron energy of 395.5 keV. In addition, the ⁸⁹Zr physical half-life is better suited to cartilage imaging (e.g., about five days are needed for the probe to accumulate in cartilage) than that of ⁶⁴Cu or ⁸⁶Y. Also, ⁸⁹Zr is safer to handle, cheaper to produce, more stable in vivo, and residualizes in tumors far more effectively than ¹²⁴I. ⁸⁹Zr emits positrons rather than single photons allows for higher resolution, quantitative imaging with PET or Cerenkevo. In preliminary chemical toxicity studies in mice, 100-200 times the contemplated dose per kilogram in man was well tolerated. No ill-effects were observed in any of the animals.

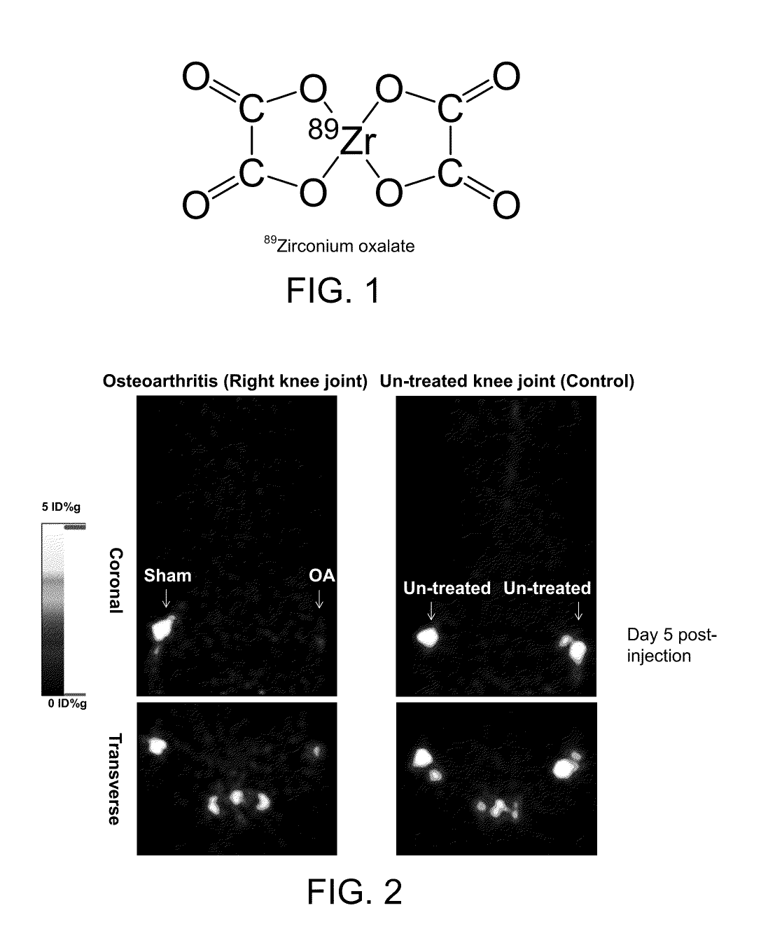

In a particular embodiment, the probe can be a carrier free ⁸⁹Zr ion. The term “carrier free” denotes a radioisotope of an element in pure form without any stable isotope carrier. The radioisotope will stay as a radioactive ion in salt solution. In an embodiment, the ⁸⁹Zr radioisotope can be dissolved in a salt buffer such as oxalate, halide (chloride), phosphate, citrate, and the like. In an embodiment, the probe can be ⁸⁹Zr oxalate, as shown in FIG. 1.

As mentioned above, an exemplary embodiment of the present disclosure includes a method of imaging a target area within a living subject (e.g., mammal such as a human) or a sample (e.g., tissue). Initially, one or more (e.g., amount and/or type) probes (e.g., radionuclide probes) are introduced into the living subject or sample. Subsequently, a low energy photon is generated by the probe and is detected as an optical signal(s). In addition, gamma rays can be detected that are indirectly derived from the probe via a positron emission. In an embodiment, the optical signal and/or the gamma rays can be correlated to the location of the probe, where the probe can be correlated to the target area. In an embodiment, the optical signal and/or the gamma rays can be used to produce an image of the target area (e.g., articular cartilage). In an embodiment, the target area can be imaged at certain time intervals (e.g., days, months, years) to access the changes in the target area (e.g., cartilage degeneration) as a function of time, treatment regime, and the like.

After the optical signal and/or gamma ray signal corresponding to the probe are obtained, the data corresponding to the optical signal and/or gamma ray signal can be processed to provide an image of the target area. In an embodiment, the image can be a planar image or can be a 3-dimensional image of the target area. In particular, the optical signal and/or gamma ray signal can be used to identify a target area from which the signals are produced (directly or indirectly). Once the signals corresponding to the radionuclide are obtained, the status of the target area or how the condition or disease affected the target area can be evaluated or monitored by comparing the image with one or more previous images and one or more subsequent images.

Embodiments of the methods of the present disclosure may be useful for diagnosing, prognosis, staging and monitoring the progression and recurrence of conditions or diseases and are expected to be widely adopted due to the higher sensitivity achieved, higher throughput, lower cost and broader user accessibility when compared to conventional imaging techniques. In an embodiment, the present disclosure also enables real-time monitoring of surgery at a surgical region of interest that may include the target area.

In an exemplary embodiment, the condition or disease can be associated with a cartilage related disease such as a cartilage degenerative disease, osteoarthritis, achondroplasia, cartilage defection, and the like. In an embodiment, the target area includes the place where cartilage is located, such as joint, rib cage and intervertebral discs, which may be afflicted with the condition or disease. Embodiments of the present disclosure can also be used in imaging the ear, the nose, the bronchial tubes and the intervertebral discs.

In an embodiment, the relative strength of the gamma rays and low energy photons detected can correspond to or are correlated with degeneration of the articular cartilage. For example, the strength or intensity of the detected gamma rays and low energy photons (or images thereof) can be compared to a standard, such as a standard of healthy cartilage, cartilage elsewhere in the subject that is healthy (or relatively more healthy) (e.g., comparing a right knee to a left knee), and/or previous scans of the same cartilage at different times. In an embodiment, the same cartilage can be examined using embodiments of the present disclosure can be performed at two or more different times (e.g., days, weeks, months, years apart), and each image can be compared to assess the degeneration, if any, of the cartilage.

In an embodiment, the probe can include, but is not limited to, a drug, a therapeutic agent, a radiological agent, a chemological agent, a small molecule drug, a biological agent (e.g., peptides, proteins, polynucleotides, DNA, RNA, antibodies, antigens, and the like), or a combination thereof, that is attached to the probe (e.g., associated with the ⁸⁹Zr radioisotope directly or indirectly). In an embodiment, the probe can inherently have an affinity (e.g., preferentially be attracted to and/or bind or exclusively attracted to and/or bind) for a target area(s) (e.g., cartilage) that may be present in the living subject or the sample. In an embodiment, the probe can include a targeting agent that has an affinity for the target(s). In an embodiment, both the probe and the targeting agent can have an affinity for the same or different target area.

In an embodiment, the targeting agent can function to cause the probe to interact (e.g., be attracted to, bond, and the like) with a target area. In an embodiment, the targeting agent can have an affinity for a cell, a tissue, a protein, DNA, RNA, an antibody, an antigen, a compound, and the like, that may be associated with a condition, disease, or related biological event, of interest of the target area. In particular, the targeting agent can function to target specific DNA, RNA, and/or proteins of interest. In an embodiment, the targeting agent can include, but is not limited to, polypeptides (e.g., proteins such as, but not limited to, antibodies (monoclonal or polyclonal)), antigens, nucleic acids (both monomeric and oligomeric), polysaccharides, sugars, fatty acids, steroids, purines, pyrimidines, ligands, aptamers, small molecules, ligands, or combinations thereof, that have an affinity for a condition, disease, or related biological event or other chemical, biochemical, and/or biological events of the condition, disease, or biological event. In an embodiment, the targeting agent can include: sequence-specific DNA oligonucleotides, locked nucleic acids (LNA), and peptide nucleic acids (PNA), antibodies, and small molecule protein receptors.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

⁸⁹Zirconium oxalate has been used to image cartilage in mice. ⁸⁹Zr has a physical half-life that is better suited to image cartilage (e.g., about five days are needed for the probe to accumulate in cartilage) than that of ⁶⁴Cu or ⁸⁶Y. Also, ⁸⁹Zr is safer to handle, cheaper to produce, more stable in vivo, and residualizes in tumors far more effectively than ¹²⁴I. ⁸⁹Zr emits positrons rather than single photons allows for high resolution, quantitative imaging with PET and/or Cerenkevo. In preliminary chemical toxicity studies in mice, 100-200 times the contemplated dose per kilogram in man was well tolerated. No ill-effects were observed in any of the animals.

In particular, FIG. 2 illustrates that the ⁸⁹Zr oxalate can specifically image the osteoarthritis and differentiate it from shamed and untreated joints (high signal in sham and untreated joints, whereas low signal in OA joints). The mice were injected with ⁸⁹Zirconium oxalate through tail vein and then imaged by small animal PET.

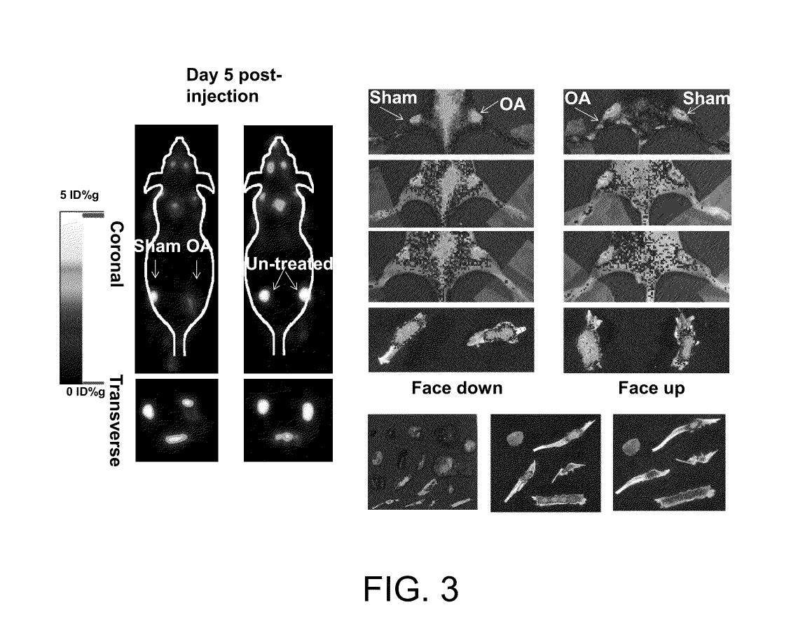

FIG. 3 illustrates the same information that the ⁸⁹Zr oxalate can specifically image the osteoarthritis and differentiate it from shamed and untreated joints (high signal in sham and untreated joints, whereas low signal in OA joints). The mice were injected with tracer through tail vein and then imaged at 5 days post-injection by small animal PET (left two columns of images) and optical imaging instrument (in vivo and ex vivo) (right two columns images).

As a result of these experiments, embodiments of the present disclosure show that probes can be used to detect the degeneration of cartilage, such as in the early stage of osteoarthritis, even before clinical symptoms and radiological changes become evident. Thus, in cases where degeneration of cartilage might be possible, imaging can be conducted early to evaluate the cartilage and compared to future images to access degeneration. This provides physician an opportunity to manage cartilage related disease prior to significant damage to the cartilage. In addition, cartilage imaging can provide boundaries that a surgeon can use during a procedure.

Methods of the present disclosure illustrates that the present probes (e.g., ⁸⁹Zr probes) provide high uptake, high sensitivity, high resolution, and high healthy cartilage/degenerated cartilage ratio. In addition, the probes show high accumulation in cartilage but clear from the body relatively quickly. Furthermore, the methods provide real-time noninvasive monitoring the radioactive materials location and distribution in subjects.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to what is being measured and the measurement technique. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

We claim at least the following:

1. A method of imaging a target area, comprising:
introducing a probe into a subject, wherein the probe is capable of emitting positrons and photons;
detecting gamma rays generated by the positrons emitted from the probe; and
detecting low energy photons generated by the probe,
wherein the origin of the gamma ray and the photons corresponds to the location of the probe, wherein the location of the probe corresponds to the location of the target area.

2. The method of claim 1, wherein the probe includes ⁸⁹Zr.

3. The method of claim 1, wherein the probe is a carrier free ⁸⁹Zr ion.

4. The method of claim 3, wherein the ⁸⁹Zr ion is dissolved in a salt buffer selected from the group consisting of: an oxalate, a halide, a phosphate, and a citrate.

5. The method of claim 4, wherein the probe is a carrier free ⁸⁹Zr ion and the target area is cartilage.

6. The method of claim 5, wherein the target area is articular cartilage.

7. The method of claim 1, wherein the probe is ⁸⁹Zr oxalate.

8. The method of claim 1, wherein the target area is articular cartilage.

9. The method of claim 9, wherein the relative strength of the gamma rays and low energy photons detected corresponds to degeneration of the articular cartilage.

10. A method of monitoring the degeneration of cartilage, comprising:
introducing a probe into a subject at a first time, wherein the probe is capable of emitting positrons and photons;
detecting gamma rays generated by the positrons emitted from the probe;
detecting low energy photons generated by the probe;
generating a first image of a target area that includes the cartilage, wherein the origin of the gamma rays and the photons corresponds to the location of the probe, wherein the location of the probe corresponds to the location of the target area;
repeating the steps above at a second time to generate a second image corresponding to the second time; and
comparing the images produced at the first time and the second time to monitor the degeneration of the cartilage.

11. The method of claim 10, wherein the cartilage is articular cartilage.

12. The method of claim 10, wherein the probe is ⁸⁹Zr oxalate.

13. The method of claim 11, wherein the probe includes ⁸⁹Zr.

14. The method of claim 11, wherein the probe is a carrier free ⁸⁹Zr ion.

15. The method of claim 11, wherein the target area is the place where cartilage located.

16. The method of claim 11, wherein the probe is a carrier free ⁸⁹Zr ion and the cartilage is the place where is cartilage located.

17. The method of claim 16, wherein the ⁸⁹Zr ion is dissolved in a salt buffer selected from the group consisting of: an oxalate, a halide, a phosphate, and a citrate.

18. The method of claim 10, wherein the relative strength of the gamma rays and low energy photons detected corresponds to degeneration of the articular cartilage, wherein a decrease in the strength of the gamma rays and low energy photons detected from the first time to the second time correlates to degeneration of the cartilage.

19. A method of imaging articular cartilage, comprising:
introducing ⁸⁹Zr oxalate into a subject, wherein the ⁸⁹Zr ion is capable of emitting positrons and photons;
detecting gamma rays generated by the positrons emitted from the ⁸⁹Zr ion; and
detecting low energy photons generated by the ⁸⁹Zr ion,
wherein the origin of the gamma ray and the photons corresponds to the location of the ⁸⁹Zr ion, wherein the location of the ⁸⁹Zr ion corresponds to the location of the articular cartilage.

20. The method of claim 19, wherein the relative strength of the gamma rays and low energy photons detected corresponds to degeneration of the articular cartilage.
